(19) United States
(12) Patent Application Publication
(10) Pub. No.: US 2024/0099601 A1
(43) Pub. Date: Mar. 28, 2024

(54) MANOMETRY SYSTEMS

(71) Applicant: Covidien LP, Mansfield, MA (US)

(72) Inventors: Jose I. Padovani, San Jose, CA (US); Benjamin J. Pagliuso, Marina Del Rey, CA (US); Christopher L. Bagley, San Jose, CA (US); Jeffrey T. Sawyer, Lakewood, CO (US); William J. Camisa, Santa Clara, CA (US); Edward L. Carcamo, Millbrae, CA (US); Mark A. Maguire, Hillsborough, CA (US); Ryan W. Bertwell, San Jose, CA (US)

(21) Appl. No.: 18/460,750

(22) Filed: Sep. 5, 2023

Related U.S. Application Data
(60) Provisional application No. 63/410,267, filed on Sep. 27, 2022.

(51) Int. Cl. A61B 5/03; A61B 5/00; A61B 5/0538

(52) U.S. Cl. CPC A61B 5/036; A61B 5/0002; A61B 5/0538; A61B 5/4233; A61B 5/4255; A61B 5/6852; A61B 2560/0223; A61B 2560/0456

(57) ABSTRACT

A manometry system includes a manometric catheter configured to sense pressure within a gastrointestinal tract, a wireless assembly configured for transmitting/receiving signals to/from the catheter, and a base unit configured to calibrate the catheter prior to usage thereof and to store and charge a wireless electronics module of the wireless assembly.

### CROSS-REFERENCE TO RELATED APPLICATIONS

This application claims the benefit of the filing date of provisional U.S. Patent Application No. 63/410,267, filed on Sep. 27, 2022.

### FIELD

This disclosure relates generally to diagnostic instruments, and more particularly, to a manometry system.

### BACKGROUND

The esophagus is a tubular organ that carries food and liquid from the throat to the stomach. Accurate measurements of physiological parameters of the esophagus under realistic swallowing conditions are valuable in diagnosing esophageal diseases such as achalasia, dysphagia, diffuse esophageal spasm, ineffective esophageal motility, and hypertensive lower esophageal sphincter (LES). When a person with a healthy esophagus swallows, circular muscles in the esophagus contract. The contractions begin at the upper end of the esophagus and propagate down toward the lower esophageal sphincter (LES). The function of the peristaltic muscle contractions is called the motility function or peristalsis.

Esophageal manometry is a medical procedure used to assess pressure and motor function of the upper esophageal sphincter (UES), the esophagus, and the lower esophageal sphincter (LES), allowing physicians to evaluate how well these muscles work to transport liquids or food from the mouth into the stomach. Similarly, anorectal manometry is a medical procedure used to assess the function of the rectal and anorectal muscles and sphincter. The results of an anorectal manometry procedure may be used to diagnose certain diseases, such as, for example, Hirschsprung's disease.

### SUMMARY

In accordance with the disclosure, a manometry system is provided that includes a manometry catheter and a wireless assembly configured to couple to the manometry catheter. The wireless assembly includes a wireless electronics module, and a catheter electronics module detachably coupled to the wireless electronics module and fixedly attached to the manometry catheter. In aspects, the wireless electronics module may be configured to communicate with a PC directly, e.g., through a wireless communication protocol. The wireless electronics module includes a processor configured to receive, via the catheter electronics module, impedance and/or pressure measurements taken by the manometry catheter, and a rechargeable battery for powering the wireless electronics module and the manometry catheter. The wireless electronics module is configured to wirelessly communicate with an external computer and transmit the impedance and/or pressure measurements from the catheter electronics module to the external computer.

In aspects, the system may further include a base unit having a charging port configured to recharge the battery of the wireless electronics module.

In aspects, the base unit may include a housing defining a calibration chamber configured to house the manometry catheter therein during calibration of a plurality of pressure sensors of the manometry catheter by the base unit.

In aspects, the base unit may include a wireless module tube extending from the housing and configured to house the wireless electronics module when the wireless electronics module is coupled to the manometry catheter, and a catheter tube each extending from the housing and configured to house a working end of the manometry catheter.

In aspects, the wireless module tube and the catheter tube may extend in parallel relation to one another.

In aspects, the base unit may include a calibration pump controller configured to calibrate the plurality of pressure sensors of the manometry catheter.

In aspects, the base unit may be configured to be detachably mounted to a surgical cart or other suitable mounting surface.

In aspects, a wireless dongle may be provided that is configured to be coupled to an external computer (e.g., a PC or a laptop) that ensures communication compatibility between the manometry system and the external computer. For example, the system may include a dedicated laptop/PC with integrated communication capability, e.g., BLUETOOTH, where the parameters are defined for secure and reliable connectivity. Alternatively, if the system is to be used with a foreign laptop/PC, the dongle may be used as part of the system to enable secure and reliable wireless communication between the system and the PC.

In accordance with another aspect of the disclosure, a base unit of a manometry system is provided and includes a housing defining a calibration chamber configured to house a manometry catheter therein during calibration of a plurality of pressure sensors of the manometry catheter by the base unit.

In aspects, the base unit may include a plurality of charging ports configured to selectively couple to and transmit power to respective wireless electronics modules.

In aspects, the plurality of charging ports may be disposed in a linear array between the wireless module tube and the catheter tube.

### BRIEF DESCRIPTION OF DRAWINGS

Various aspects of the disclosure are described herein with reference to the drawings wherein:

FIG. 1 is a perspective view illustrating a manometry system including a control station, a base unit, a dongle, a plurality of wireless electronics modules, and a catheter in accordance with the disclosure;

FIG. 2 is a perspective view illustrating three types of manometric catheters of the system of FIG. 1 for selective attachment to the wireless electronics module;

FIG. 3 is a perspective view illustrating the wireless assembly of the system of FIG. 1 including the wireless electronics module and a catheter electronics module;

FIG. 4 is a perspective view illustrating the wireless electronics module of the wireless assembly of FIG. 3;

FIG. 5 is a perspective view illustrating a catheter electronics module of the wireless assembly of FIG. 3;

FIG. 6A is a block diagram illustrating components of the manometry system of FIG. 1 including a computer, the dongle, the base unit, the wireless electronics module, and the catheter;

FIG. 6B is a block diagram illustrating internal components of the wireless assembly of the manometry system of FIG. 1 and a connected catheter;

FIG. 7 is a front view illustrating the base unit of the manometry system of FIG. 1;

FIG. 8 is a perspective view illustrating the base unit of FIG. 7;

FIG. 9 is a cross-sectional view illustrating an inside of the base unit of FIG. 7; and

FIG. 10 is a perspective view illustrating the base unit of FIG. 7 mounted to a side of a surgical cart.

### DETAILED DESCRIPTION

The disclosed manometry system and components thereof will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

Esophageal manometry is a medical procedure used to assess pressure and motor function of the esophagus, allowing physicians to evaluate how well the muscles in the esophagus work to transport liquids or food from the mouth into the stomach. To perform this procedure, the manometry system operates in conjunction with a manometric catheter placed in the esophagus of a patient to record pressure and/or impedance data over a period of time using various sensors placed on the catheter. The data is analyzed using analysis software to evaluate causes of, and help diagnose conditions such as, gastric reflux, dysphagia, functional chest pain, achalasia, and hiatal hernia.

The manometry system obtains high resolution and/or three-dimensional (3D) mapping of pressure levels within the tubular organs of the human gastrointestinal tract, and optionally pressure in conjunction with impedance levels, within the tubular organs of the human gastrointestinal tract which may include the pharynx, esophagus, proximal gut (stomach/duodenum), anus, and rectum. The manometry system is used in a medical clinical setting to acquire the pressure and impedance levels and store the corresponding data for visualization and analysis using the software. Esophageal manometry is used as an example; the systems and methods of the disclosure are applicable to other forms of manometry systems, for example, an anorectal manometry system.

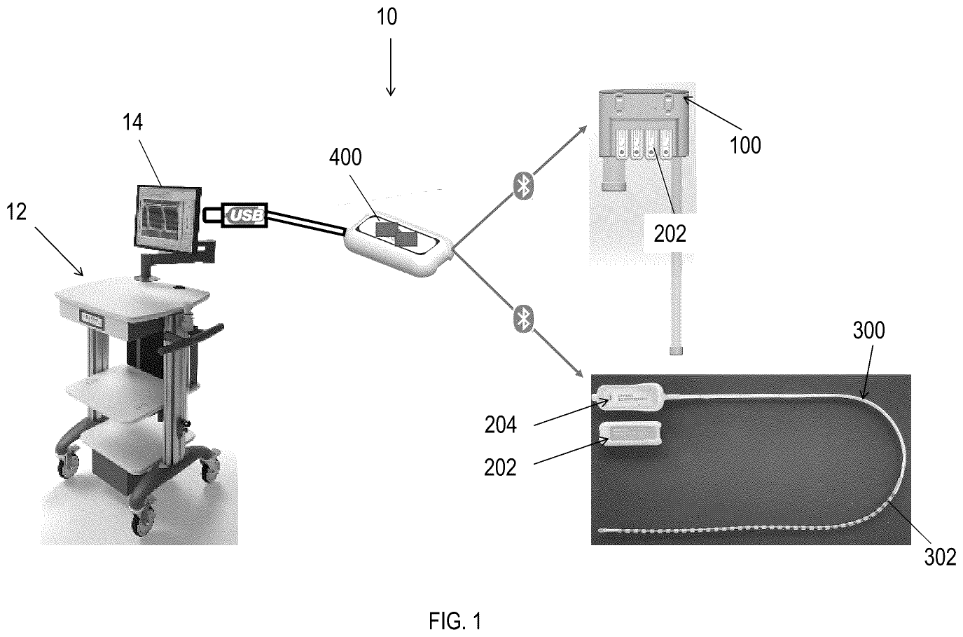

FIG. 1 illustrates a manometry system 10. The manometry system 10 generally includes a surgical cart 12 or other suitable external support surface, a computer 14 (e.g., a personal computer, laptop or a tablet), a base unit 100 supporting a plurality of wireless electronics modules 202, a manometric catheter or probe 300 supporting a catheter electronics module 204, and in some aspects a wireless dongle 400. When a selected wireless electronics module 202 is connected to a catheter electronics module 204 of a catheter 300, the combined wireless electronics module 202 and the catheter electronics module 204 together form a wireless assembly 200 (FIG. 3) configured to execute software for data acquisition and processing, and provide compatibility with various manometric catheter 300 configurations depending on the application (esophageal/anorectal manometry), size, and catheter diameter.

The manometry system 10 enables full evaluation of the motor functions of an esophagus. The system 10 allows for enhanced sensitivity that provides useful information to support diagnosis of conditions like dysphagia, achalasia, and hiatal hernia. By precisely quantifying the contractions of the esophagus and its sphincters, this procedure helps provide a more complete esophageal pressure profile of the patient.

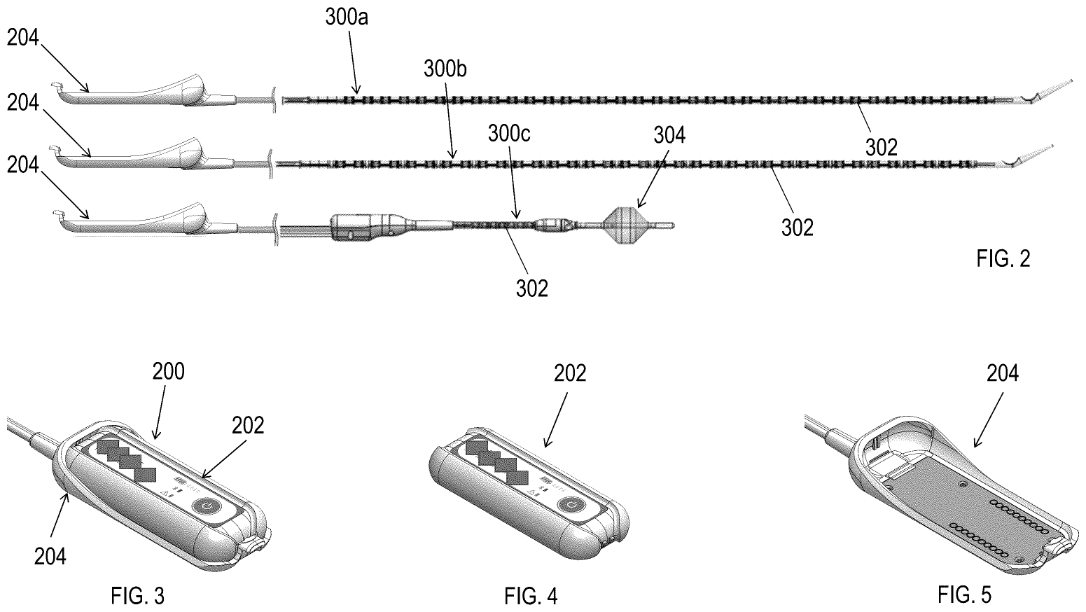

With reference to FIGS. 1 and 2, esophageal pressure measurement, or manometry, as well as electrical impedance, can be used to assess motility function of the esophagus and bolus transit dynamics in the esophagus. The manometric catheter 300 includes a plurality of sensor assemblies 302 (e.g., pressure sensors) located along its length. Details regarding exemplary types of sensor assemblies for use in the system of the present disclosure may be found in WO2022/086838, filed on Oct. 18, 2021, the entire contents of which being incorporated by reference herein. The manometric catheter 300 may be inserted into the esophagus, typically reaching the lower esophageal sphincter (LES) and extending into the stomach of a patient, with the pressure sensors 302 positioned at the LES and at a plurality of other specific points along the length of the esophagus at predetermined distances above the LES. The LES is a muscle that separates the esophagus from the stomach. It acts as a valve that normally stays tightly closed to prevent contents in the stomach from backing up into the esophagus.

During a procedure, the patient swallows (e.g., with or without liquid) with the manometric catheter 300 placed in the esophagus. The esophageal pressure at the sensor assemblies 302 can be measured and used as an indication of the magnitude and sequence of the peristaltic contractions. In addition, because the positions of the sensor assemblies 302 are known, the velocity of the peristaltic motion can also be ascertained from the location of the peak pressure, or onset of pressure rise, at each location as a function of time. The test can be repeated a number of times to obtain a set of pressure and velocity values, a statistical analysis of which may be used for diagnostic purposes.

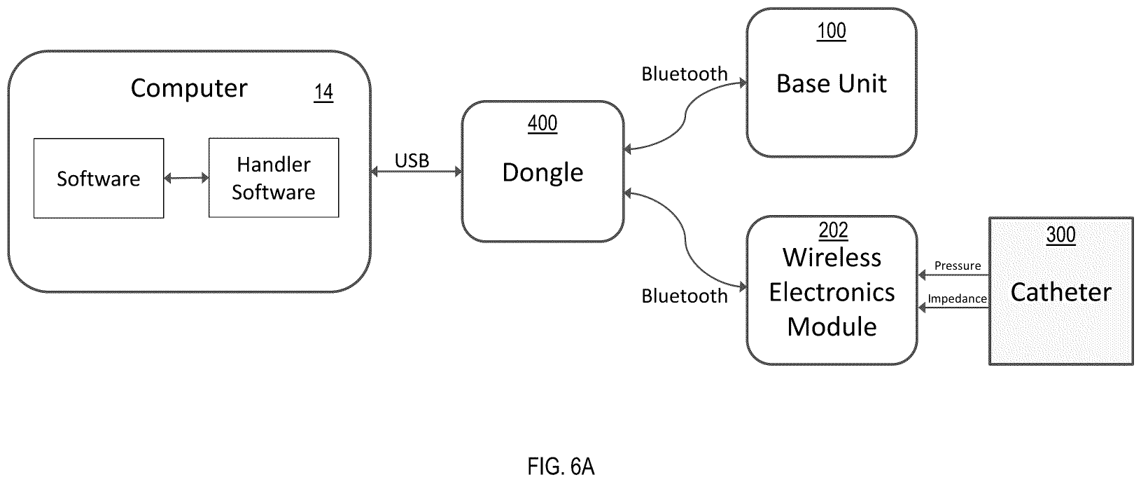

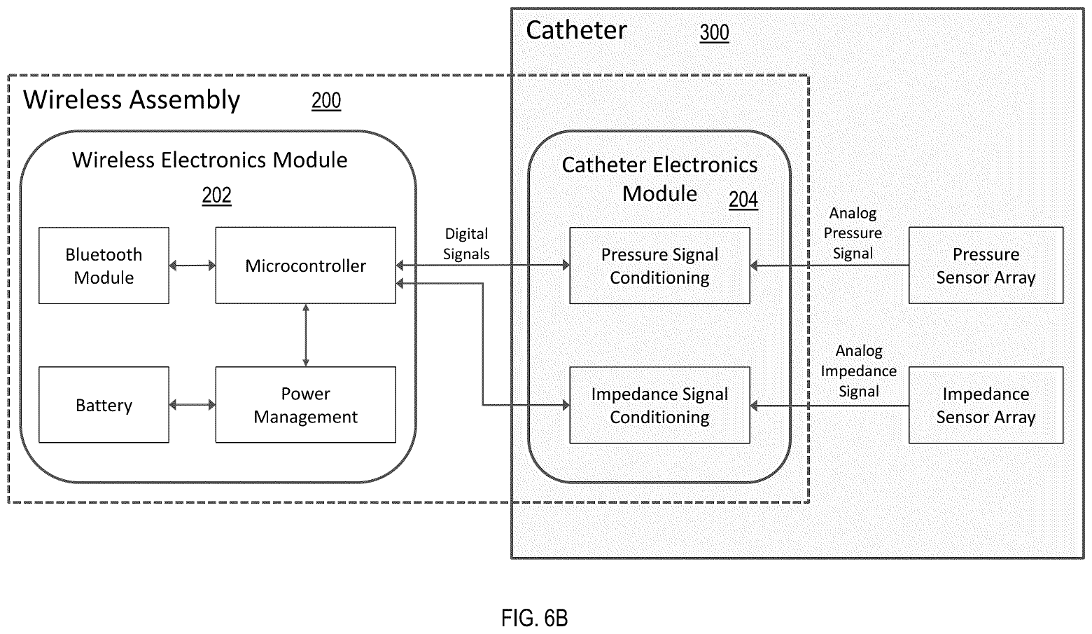

High-resolution manometry involves the collection of data with a catheter having closely spaced sensors. Such high-resolution data enables spatiotemporal contour plots visualization of contractile pressure physiology. Products such as ManoScan™ data acquisition software and ManoView™ data analysis software may be used to aid in acquiring and visualizing high-resolution manometry data. It is contemplated that the aforementioned software may be installed on the computer 14. The software receives data from the wireless assembly 200 via the dongle 400 (FIG. 6A), or directly from the computer 14 (FIG. 1).

The manometric catheter 300 may include other sensors (not labeled) such as impedance sensors. High-resolution impedance measurements provide for spatiotemporal plotting of bolus movement. Electrical impedance at a plurality of points in the esophagus can be used to detect and monitor the movement of a bolus through the esophagus. A bolus of water or food will have different electrical impedance than the non-filled esophagus, so a change in impedance in the esophagus indicates the presence of a bolus. Therefore, the manometric catheter 300 positioned in the esophagus with a plurality of impedance and/or acidity sensors dispersed along its length can be used to detect and monitor the bolus transit, i.e., the movement of a bolus through the esophagus.

In aspects, as shown in FIG. 2, the system 10 may include three different types of manometry catheters 300 each with a different functionality. For example, the system 10 may include a first esophageal manometry catheter 300a configured to take pressure measurements only, a second esophageal manometry catheter 300b configured to take pressure and impedance measurements, and a third anorectal manometry catheter 300c configured to take pressure measurements utilizing the plurality of pressure sensors 302 and a detachable, inflatable balloon 304. Each of the catheters 300 support (e.g., are fixedly secured to) a distal end of the catheter electronics module 204.

With reference to FIGS. 3-6B, the wireless assembly 200 is configured to be in wireless communication with the dongle 400 to transmit data (e.g., impedance and pressure measurements of the catheter 300) to the computer 14 via the dongle 400. In aspects, the computer 14 may be configured to wirelessly operate a calibration pump controller system 112 (FIG. 9) in the base unit 100.

With reference to FIG. 6B, the catheter electronics module 204 is a connector cradle integrated into the proximal end of the catheter 300 and is configured to detachably couple the wireless electronics module 202 to a proximal end of a selected one of the catheters 300. The catheter electronics module 204 includes impedance and pressure conditioning electronics (FIG. 6B), is waterproof, disposable, and high level disinfection compatible. The wireless electronics module 202 has a wireless communication function for wirelessly communicating with the computer 14 (e.g., via the dongle 400) and the catheter 300 via the catheter electronics module 204. For example, the wireless electronics module 202 is BLUETOOTH® enabled, reusable, and wiping compatible, and includes a rechargeable battery. The wireless electronics module 202 of the wireless assembly 200 further includes a processor or microcontroller and power management. In various aspects of the disclosure, the processor may be any suitable type of processor such as, without limitation, a digital signal processor, a microprocessor, an ASIC, a field-programmable gate array (FPGA), or a central processing unit (CPU).

The dongle 400 (FIGS. 1 and 6A) may be a BLUETOOTH®-enabled dongle. In aspects, the dongle 400 is configured to wirelessly communicate with the connected catheter (e.g., 300a, 330b, or 300c) of the manometry system 10. In aspects, the dongle 400 may be configured to wirelessly communicate with the components of the manometry system 100 using various radio frequency protocols such as near field communication, radio frequency identification “RFID,” BLUETOOTH®, etc. The dongle 400 connects to the computer 14 (e.g., via USB) and thereby provides wireless communication to the base unit 100. The dongle 400 also connects to the assembly 200.

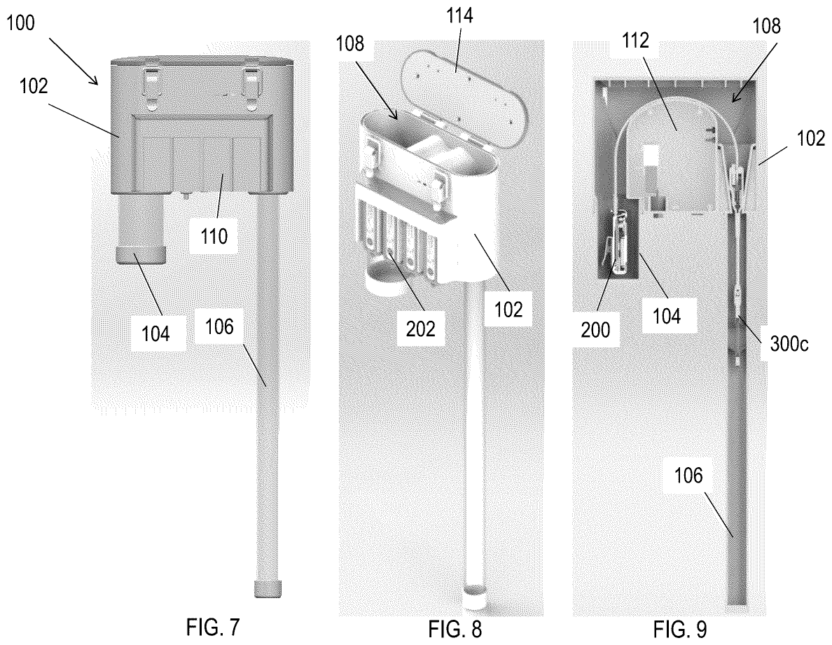

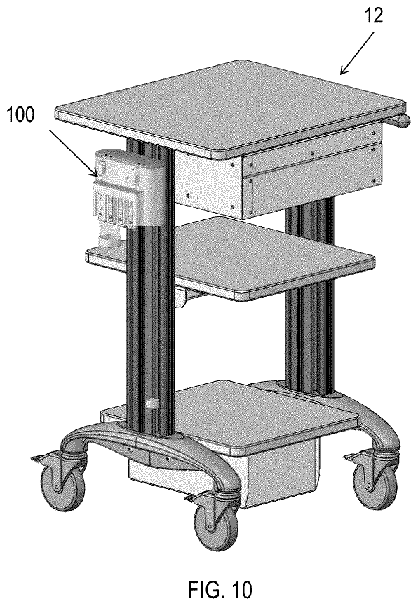

With reference to FIGS. 7-10, the base unit 100 of the system 10 may be supported on a table or detachably connected to the surgical cart 12 (FIG. 1) or other suitable support surface and includes a housing 102, a pump controller module 112, a wireless module tube 104 extending distally from the housing 102, and a catheter chamber tube 106 extending distally from the housing 102. The pump controller module 112 may be configured to receive wall power through a fixed or detachable electrical power adapter and cable. The housing 102 defines a chamber 108 therein and a plurality (e.g., four) charging slots or ports 110 configured to detachably secure to a respective wireless electronics module 202. As such, the batteries of each of the wireless electronics modules 202 may be recharged by the base unit 100 when not being used. Each of the charging ports 110 may have a power connector that shares power with a pump controller system 112. When connected to the charging ports 110, the battery level indicator on the wireless electronics modules 202 is exposed and the connector of the wireless electronics module 202 is concealed.

The chamber 108 of the housing 102 is configured to receive and calibrate either of the three catheters 300a, 300b, or 300c, and supports the pump controller system 112 therein. The wireless module tube 104 and the catheter tube 106 extend in parallel relation to one another with the charging ports 110 positioned therebetween. The wireless module tube 104 is configured to house the wireless assembly 200 that is coupled to the catheter 300, and the catheter tube 106 is configured to house the working end of the catheter 300 (e.g., the flexible body having the pressure sensors 302) during calibration of the catheter 300. The base unit 100 may be BLUETOOTH® enabled to allow for communication with, for example, the computer 14 or the dongle 400. The base unit 100 may further include a lid 114 pivotably coupled to the housing 102 for selectively covering the chamber 108, and a mount (not labeled) for detachably coupling the base unit 100 to a wall or the cart 12. The lid 114 forms an air-tight seal to fully enclose the catheter 300 in the chamber 108 and allows for pressurization therein during calibration of the catheter 300.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A manometry system, comprising:
a manometry catheter; and
a wireless assembly including:
a wireless electronics module configured to be in wireless communication with an external computer; and
a catheter electronics module connected to the manometry catheter and configured to detachably connect to the wireless electronics module, wherein the wireless electronics module is configured to receive, via the catheter electronics module, at least one of impedance or pressure measurements taken by the manometry catheter.

2. The manometry system according to claim 1, further comprising a base unit including a charging port configured to recharge a battery of the wireless electronics module.

3. The manometry system according to claim 2, wherein the base unit includes a housing defining a calibration chamber configured to hermetically seal in its entirety the manometry catheter therein during pressure calibration of a plurality of pressure sensors of the manometry catheter in conjunction with the base unit.

4. The manometry system according to claim 3, wherein the base unit includes a wireless module tube extending from the housing and configured to house the wireless electronics module when the wireless electronics module is coupled to the manometry catheter, and a catheter tube each extending from the housing and configured to house a working end of the manometry catheter.

5. The manometry system according to claim 4, wherein the base unit includes a calibration pump controller configured to calibrate the plurality of pressure sensors of the manometry catheter.

6. The manometry system according to claim 1, further comprising a dongle configured to wirelessly communicate with the wireless electronics module for wireless receiving the at least one of the impedance or pressure measurements.

7. A base unit of a manometry system, the base unit comprising:
a housing defining a calibration chamber configured to house a manometry catheter therein during calibration of a plurality of pressure sensors of the manometry catheter by the base unit; and
a plurality of charging ports configured to selectively couple to and transmit power to a respective wireless electronics module.

8. The base unit according to claim 7, further comprising:
a wireless module tube extending from the housing and configured to house the wireless electronics module when the wireless electronics module is coupled to the manometry catheter; and
a catheter tube each extending from the housing and configured to house a working end of the manometry catheter.

9. The base unit according to claim 8, further comprising a calibration pump controller configured to calibrate the plurality of pressure sensors of the manometry catheter.

10. The base unit according to claim 8, wherein the wireless module tube and the catheter tube extend in parallel relation to one another.

11. The base unit according to claim 10, wherein the plurality of charging ports are disposed in a linear array between the wireless module tube and the catheter tube.

12. The base unit according to claim 7, wherein the base unit is configured to be detachably mounted to an external surface.
